(19) United States
(12) Patent Application Publication
(10) Pub. No.: US 2023/0215149 A1
(43) Pub. Date: Jul. 6, 2023

(54) SYSTEM FOR GENERATION OF USER-CUSTOMIZED IMAGE IDENTIFICATION DEEP LEARNING MODEL THROUGH OBJECT LABELING AND OPERATION METHOD THEREOF

(71) Applicant: XII Lab, Gangnam-gu Seoul (KR)

(72) Inventors: Woo Yung LEE, Gangnam-gu, Seoul (KR); Dae Su CHUNG, Gangnam-gu, Seoul (KR); Se Hun KIM, Dobong-gu, Seoul (KR)

(73) Assignee: XII Lab, Gangnam-gu Seoul (KR)

(21) Appl. No.: 18/010,480

(22) PCT Filed: Jul. 2, 2020
(86) PCT No.: PCT/KR2020/008631

§ 371 Date: Mar. 21, 2023

(30) Foreign Application Priority Data
Jun. 16, 2020 (KR) 10-2020-0072769

(51) Int. Cl. G06V 10/774; G16H 30/40

(52) U.S. Cl. CPC G06V 10/7753; G16H 30/40; G06V 2201/031

(57) ABSTRACT

A deep learning system establishes a simple process of generating a deep learning model, and provides an intuitive, natural and easy interaction in performing feedback on image input, manual labelling and automated labelling required for the above-described operations. Therefore, a user without expertise in deep learning can have an opportunity to directly generate and use a user-customized image identification deep learning model for identifying a desired object to be identified.

### TECHNICAL FIELD

The present disclosure relates to a system for generating a user-customized deep learning model through object labelling and an operation method thereof.

### BACKGROUND

Unless otherwise indicated herein, the materials described in this section are not prior art to the claims in this application and are not admitted as prior art by inclusion in this section.

Since image analysis technology has advanced, deep learning models are being applied to various methods for identifying an object in an image. As for the method using a deep learning model to identify an object in an image, whether a dataset for learning is sufficient is more important than a learning method and algorithm of the deep learning model. Conventional datasets of deep learning models for identifying an object in an image are constructed to identify limited objects such as persons or animals. Therefore, it is difficult to identify an object in an image by using a deep learning model in various fields.

Therefore, users without expertise in deep learning have no choice but to simply use an existing deep learning model. Accordingly, the types of identifiable objects are limited, and satisfactory performance of the identification function cannot be achieved.

Also, in constructing a dataset for deep learning model, platforms such as Mechanical Turk may provide an interface that enables a general user, not a developer, to perform a labelling operation. However, the labelling operation is just one step in generating a deep learning model. Thus, the user without expertise in deep learning cannot generate a deep learning model capable of identifying a desired object without the help of a deep learning developer.

### SUMMARY

The present disclosure is intended to solve the above-described problems and/or various other problems and may provide a user-customized image identification deep learning system that enables even a user without expertise in deep learning to construct a dataset of a deep learning model for identifying a desired object.

More specifically, the present disclosure is intended to provide a user-customized image identification deep learning system that provides a user interface configured to enable a user to directly upload an image and perform labelling on a certain object in the uploaded image.

Further, the present disclosure is intended to provide a user-customized image identification deep learning system that includes a user interface configured to enable a user to perform feedback so that a deep learning model generated using a dataset constructed by the user can secure sufficient reliability.

That is, the user-customized image identification deep learning system of the present disclosure is intended to provide a platform that enables an expert in any other field without expertise deep learning to easily and efficiently construct a deep learning model.

According to an aspect of the present disclosure, an operation method of a user-customized image identification deep learning system may include: a process of receiving at least one first image in response to a request from a user device; a process of performing manual labelling based on a user input on the at least one first image from the user device and storing the manually labelled at least one first image as a first dataset; a process of generating a first deep learning model based on the first dataset; a process of receiving at least one second image in response to a request from the user device; a process of performing automated labelling on the at least one second image by using the first deep learning model; a process of storing at least one of the automatically labelled at least one second image as a second dataset based on a result of feedback on the automatically labelled at least one second image from the user device; and a process of generating a second deep learning model based on the first dataset and the second dataset.

According to an embodiment, the operation method of a user-customized image identification deep learning system may further include a process of measuring an accuracy of the second deep learning model.

According to an embodiment, the operation method of a user-customized image identification deep learning system may further include a process of updating the first dataset or the second dataset when the accuracy of the second deep learning model is measured to be equal to or lower than a reference level.

According to an embodiment, the operation method of a user-customized image identification deep learning system may further include a process of providing the user device with a first user interface including an image input button configured to receive the at least one first image, an image display region configured to display the at least one first image, a labelling tool configured to provide manual labelling to each of the at least one first image, and a storage button configured to request storage of the manually labelled at least one first image as a first dataset.

According to an embodiment, in the operation method of a user-customized image identification deep learning system, the process of receiving at least one first image may include a process of receiving information about an access route to an image providing device.

According to an embodiment, in the operation method of a user-customized image identification deep learning system, the process of receiving at least one first image may include a process of receiving an image stored in the user device.

According to an embodiment, the operation method of a user-customized image identification deep learning system may further include a process of providing the user device with a first user interface including an image input button configured to receive the at least one second image, an image display region configured to display the automatically labelled at least one second image, and a feedback input button configured to receive feedback on the automatically labelled at least one second image.

According to an embodiment, the operation method of a user-customized image identification deep learning system may further include a process of providing a third user interface including an image input button configured to receive a third image, and an image identification request button configured to request application of the second deep learning model to the third image.

According to an embodiment, in the operation method of a user-customized image identification deep learning system, the third user interface may further include an accuracy display region configured to display the accuracy of the second deep learning model.

The foregoing summary is illustrative only and is not intended to be in any way limiting. In addition to the illustrative embodiments and features described above, further embodiments and features will become apparent by reference to the drawings and the following detailed description.

### BRIEF DESCRIPTION OF THE DRAWINGS

The foregoing and other features of the present disclosure will be described in detail with reference to the accompanying drawings. Understanding that these drawings depict only several examples in accordance with the disclosure and are, therefore, not to be considered limiting of its scope, the disclosure will be described with additional specificity and detail through use of the accompanying drawings, in which:

FIG. 1 is a configuration view of a user-customized image identification deep learning system according to an embodiment of the present disclosure;

FIG. 2 shows an example of a process for generating a user-customized image identification deep learning model according to at least some embodiments of the present disclosure;

FIG. 3 shows a specific process of a manual labelling operation that is performed in the user-customized image identification deep learning system according to at least some embodiments of the present disclosure;

FIG. 4 shows an example of a user interface to which the user-customized image identification deep learning system provides a manual labelling operation according to at least some embodiments of the present disclosure;

FIG. 5 shows another example of a user interface to which the user-customized image identification deep learning system provides a manual labelling operation according to at least some embodiments of the present disclosure;

FIG. 6 shows a process of an automated labelling operation that is performed in the user-customized image identification deep learning system according to at least some embodiments of the present disclosure;

FIG. 7 shows an example of a user interface to which the user-customized image identification deep learning system provides an automated labelling operation according to at least some embodiments of the present disclosure;

FIG. 8 shows a specific example of a deep learning user interface provided by the user-customized image identification deep learning system and displayed on a user device according to at least some embodiments of the present disclosure; and

FIG. 9 shows an example of a computer program product configured to operate a system for generating a user-customized image identification deep learning model according to at least some embodiments of the present disclosure.

### DETAILED DESCRIPTION

The terms used herein are used only to describe specific examples, but do not intend to limit the present disclosure. A singular expression includes a plural expression unless it is clearly construed in a different way in the context. All terms including technical and scientific terms used herein have the same meaning as commonly understood by a person with ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. In some cases, even terms defined in the present disclosure should not be interpreted as excluding embodiments of the present disclosure.

The foregoing features and other features of the present disclosure will be sufficiently apparent from the following descriptions with reference to the accompanying drawings. These drawings merely illustrate several embodiments in accordance with the present disclosure. Therefore, they should not be understood as limiting the present disclosure. The present disclosure will be described in more detail with reference to the accompanying drawings.

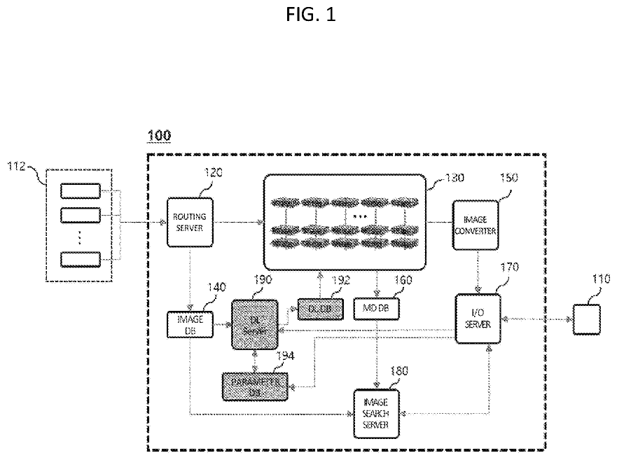

FIG. 1 is a configuration view of a user-customized image identification deep learning system according to an embodiment of the present disclosure. Referring to FIG. 1, a user-customized image identification deep learning system 100 (hereinafter, referred to as “system 100”) may provide a user device 110 with a user interface configured to provide a deep learning model for user-customized image identification. A user may use user device 110 to generate and use a user-customized image identification deep learning model through the user interface provided by system 100. According to an embodiment, user device 110 may be a computing device capable of wired/wireless communication. For example, user device 110 may include a portable device such as a cell phone, a smart phone, a PDA, a tablet and a laptop, or a non-portable computing device such as a desktop and a server.

System 100 may be configured to receive an input image as a dataset from an external input image providing device 112. System 100 may provide user device 110 with a result image obtained by processing the input image with a deep learning model. A communication linkage between system 100 and input image providing device 112 and/or user device 110 may be established in various wired or wireless ways. A network that enables the communication linkage may include, for example, a radio frequency (RF) network, a third generation partnership project (3GPP) network, a long term evolution (LTE) network, a world interoperability for microwave access (WIMAX) network, the Internet, a local area network (LAN), a wireless LAN, a wide area network (WAN), a personal area network (PAN), a Bluetooth network, a near field communication (NFC) network, a satellite broadcasting network, an analog broadcasting network, and a digital multimedia broadcasting (DMB) network, but is not limited thereto.

In an embodiment, input image providing device 112 may include a plurality of input image generating devices. The input image generating device may include an imaging device such as a general camera. Also, the input image generating device may be a device, such as a synthetic image generator, that generates a synthetic image instead of an actual image obtained through an imaging device. Each of such various input image generating devices constitutes a single image channel, and input images generated by each channel are provided as a dataset to system 100. In an embodiment, input image providing device 112 may include a database configured to store an input image. The database may be a component included in user device 110, and may include a separate web server or cloud network server. The user may use user device 110 to provide an input image stored in input image providing device 112 to system 100. For example, user device 110 may provide information about an access route and/or access authority to input image providing device 112 by system 100. Hereinafter, the route through which image providing device 112 provides an input image to system 100 may be referred to as a channel.

System 100 shown in FIG. 1 may include a routing server 120, an image analyzing server cluster 130, an image database 140, an image converter 150, a metadata database 160, an I/O server 170, an image search server 180, a deep learning server 190, a deep learning database 192 and a parameter database 194. Herein, I/O server 170 may be omitted in some cases. In this case, image converter 150 may directly provide processed image data to user device 110 through the above-described communication linkage, and user device 110 may directly perform an image search through the communication linkage with image search server 180.

Routing server 120 may receive input image from each channel of image providing device 112 and store the input image as original image data in image database 140. The original image data stored in image database 140 may be provided to user device 110 in response to the user's request for search.

Also, routing server 120 may route processing of images from a specific channel to a specific server in image analyzing server cluster 130 according to characteristics of each channel of image providing device 112.

Image analyzing server cluster 130 is composed of a plurality of image analyzing servers, and each image analyzing server may be equipped with one or more high-specification GPUs for high-quality image analysis. Also, each image analyzing server may be designed to be suitable for analysis of a specific image. For example, each image analyzing server may be classified into an image analyzing server suitable for recognizing and processing a person, an image analyzing server suitable for recognizing and processing a car, and the like depending on an object included in an image. Further, each image analyzing server may be designed to be suitable for processing image data of a specific situation, and may be classified into, for example, an image analyzing server suitable for processing image data with generally low brightness, such as an image taken by a camera installed in a tunnel, an image analyzing server suitable for processing image data with generally high brightness, such as an image taken by a camera installed outdoors, and the like. Furthermore, each image analyzing server may be designed to be suitable for processing image data according to characteristics of each channel, such as the type of the channel. For example, each image analyzing server may be classified into an image analyzing server with a fixed channel suitable for processing high-quality image data, such as a CCTV, an image analyzing server with a mobile channel suitable for processing low-quality image data, such as a drone, and the like.

Each image analyzing server of image analyzing server cluster 130 may analyze image data from a specific channel allocated by the routing server, extract metadata of the image data and then store the metadata in metadata database 160. Also, each image analyzing server may generate inverse features from the extracted metadata and store the inverse features in metadata database 160. The metadata may include information in which an object recognized in an image is tagged according to time and type. For example, an object included in an image may include various types of objects such as persons, cars, etc. The metadata may include a matrix data structure including at least one row corresponding to the type of the object identified in the image and at least one column corresponding to the time when the object is displayed in the image.

The inverse features may be generated based on the metadata. As the inverse features, information about a channel that photographs a corresponding object is listed in chronological order based on each object. The inverse features broadly classify objects into a person, a car, etc. and further classify the objects classified as persons, and may include detailed information of the objects. For example, the detailed information included in the inverse features includes information about the location of the corresponding channel, a timestamp indicating the time at which the corresponding object was photographed, and the location of the original image data in which the corresponding object was photographed, but is not limited thereto.

In an embodiment, each image analyzing server of image analyzing server cluster 130 may process an input image by applying an image recognition algorithm suitable for a channel for image data to be processed. In this case, the image analyzing server may retrieve metadata of a corresponding channel from a channel meta-database regarding attributes of each channel and process an input image by applying an image recognition algorithm suitable for the channel metadata. The channel metadata may include camera ID, camera IP, encoding type (e.g., H.264, H.265, etc.), camera type (e.g., CCTV, drone, etc.), image resolution (e.g., HD, 4K, etc.), imaging device type (e.g., fixed, mobile, etc.), content category (e.g., parking lot, downtown street, etc.), camera location, camera height, tilt angle, pan angle, number of decoding frames per second, use, and the like, but may not be limited thereto. The channel meta-database, which is a set of channel metadata, may be stored in the form of metadata database 160 or a separate database, and may be searched and used by each image analyzing server of image analyzing server cluster 130.

Meanwhile, the input image from each channel recognized and analyzed by each image analyzing server of image analyzing server cluster 130 is provided to image converter 150, and image converter 150 converts the recognized and analyzed input image into a predetermined format suitable for transmission to and display on user device 110. Herein, the predetermined format may be previously set by the user of user device 110, and parameters for determining the predetermined format may be stored in parameter database 194. The input image converted into the predetermined format by image converter 150 may be provided to user device 110 through I/O server 170.

The user of user device 110 accessing system 100 from the outside may make a request for search for specific image data to system 100 through user device 110 by using a specific search query. In an embodiment, the user of user device 110 may specify an object to be searched through the picture displayed on user device 110 and make a search query. The search query may include a tag or label of a specific object, a channel that provides an image including the object, a place and/or time period thereof.

I/O server 170 of system 100 may receive the search query from user device 110 and provide the search query to image search server 180. If the above-described I/O server 170 is omitted, the search query from user device 110 may be directly provided to image search server 180.

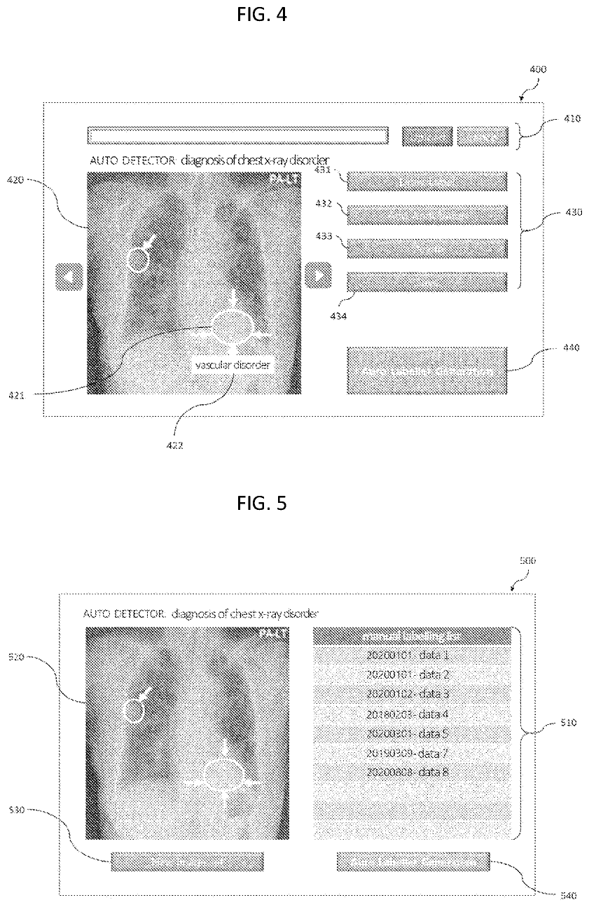

Image search server 180 may first search metadata database 160 by using the search query transmitted from user device 110. In this case, image search server 180 may specify a search target object included in the search query by using a tag, label or thumbnail of the object to be searched and obtains a channel that takes an image including the object and a photographing time of the image from metadata database 160. Image search server 180 may search image database 140 for original image data based on the obtained channel and photographing time of the image including the object, and may provide the searched original image data to user device 110 through I/O server 170. A specific example of searching metadata database 160 and image database 140 for original image data suitable for the search query from user device 110 and providing the same by image search server 180 will be described below in more detail with reference to FIG. 4.

Meanwhile, image database 140 may be configured as a relational database, and may be configured as a NoSQL database in which the schema is not previously defined in some embodiments.

According to an embodiment of the present disclosure, if image database 140 is configured as a NoSQL database, it may be stored in HBase. HBase classified as a column-oriented NoSQL database may store a number of columns in a row, and image database 140 of the present disclosure may generate an input image input from a specific channel in a row without limitation to the number of columns by using this attribute of HBase.

As a non-limiting example, image database 140 may record an input image received from a specific channel in a row on a daily basis, and the input image may be generated as an individual file every second in the row. In this case, a total of 86,400 (60 seconds×60 minutes×24 hours) files, i.e., columns, may be generated in a row. If image database 140 is configured in this way, it is only necessary to search a row of a specific date for an image without a need to search all the rows. Accordingly, the search efficiency can be improved.

In system 100 shown in FIG. 1, various image data received from image providing device 112 that provides various input images may be configured as image database 140 for efficient search. Routing server 120 may allocate the image data to a suitable image analyzing server of image analyzing server cluster 130 according to the type or characteristics of input image to perform image analysis and recognition. Thus, the recognition efficiency can be improved. Since routing server 120 allocates the image data to a suitable image analyzing server of image analyzing server cluster 130 according to the type or characteristics of input image, the analyzed and recognized image data may be converted into a format satisfying the requirements of the user through image converter 150 and provided to user device 110.

Further, system 100 shown in FIG. 1 can extract metadata from the image data for object search and store inverse features generated from the metadata in metadata database 160. Metadata database 160 enables efficient specification of a channel and time of an image including a specific object according to a search query from user device 110 and also enables the channel and time to be quickly searched from image database 140.

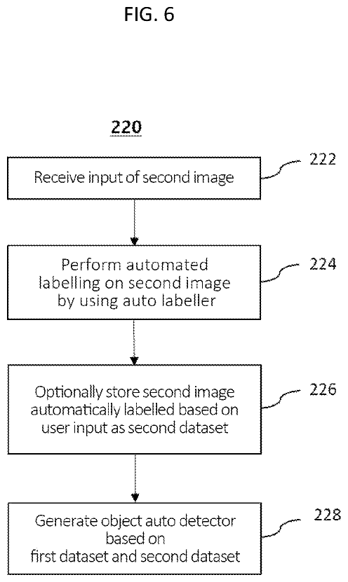

In another embodiment, converter 150 may be provided in user device 110 outside system 100. In this case, image converter 150 may receive image data analyzed and recognized by image analyzing server cluster 130 of system 100 through I/O server 170. Image converter 150 may convert the analyzed and recognized image data into a format previously set by the user of user device 110 and then provide it to user device 110, and user device 110 may display the same. According to an embodiment, the format previously set by the user may be a screen or graphic user interface to be displayed. This graphic user interface may include, for example, a part showing real-time images from a plurality of channels, a part showing the state of the spaces covered by the respective channels, and a part showing the movement paths of objects photographed by each channel as an analysis result of the channel. The graphic user interface will be described in detail later with reference to FIG. 6.

Deep learning server 190 may analyze original image data stored in image database 140 to generate an image analysis model or image recognition algorithm, and the generated image analysis model or image recognition algorithm may be stored as a deep learning result or learning data result in deep learning database 192. Further, the image analysis model or image recognition algorithm generated by deep learning server 190 may be used by each image analyzing server of image analyzing server cluster 130. Each image analyzing server of image analyzing server cluster 130 may search learning data result database 192 for an image analysis model or image recognition algorithm suitable for a specific channel allocated to itself by routing server 120 and/or a specific object and may retrieve it for application to image analysis and object recognition.

In an embodiment related to deep learning of original image data and generation of a result image analysis model, deep learning server 190 may divide and analyze the original image data stored in image database 140 by predetermined categories. For example, deep learning server 190 may analyze each object according to the type of the object photographed in the original image data in consideration of characteristics and surrounding conditions of the object, and may generate an image analysis model or image recognition algorithm therefor. In another example, deep learning server 190 may analyze an object according to a channel that records original image data in consideration of the type or characteristics of the channel, and may generate an image analysis model or image recognition algorithm therefor. In this case, deep learning server 190 may use metadata of the channel, and the channel metadata may include camera ID, camera IP, encoding type (e.g., H.264, H.265, etc.), camera type (e.g., CCTV, drone, etc.), image resolution (e.g., HD, 4K, etc.), imaging device type (e.g., fixed, mobile, etc.), content category (e.g., parking lot, downtown street, etc.), camera location, camera height, tilt angle, pan angle, number of decoding frames per second, use, and the like, but may not be limited thereto. The channel meta-database, which is a set of channel metadata, may be stored in the form of a separate database, and may be used to generate an image analysis model or image recognition algorithm by deep learning server 190.

The image analysis model or image recognition algorithm generated by deep learning server 190 may be stored by category in learning data result database 192. For example, learning data result database 192 may store image recognition algorithms for person, car, pet, etc. depending on the type of an object, image recognition algorithms for street, park, parking lot, etc. depending on positional characteristics of a channel, and image recognition algorithms for CCTV, drone, etc. depending on the type of a channel.

The image analysis model or image recognition algorithm stored in database 192 as a deep learning model automatically trained internally for specific data may further include an image analysis model or image recognition algorithm generated externally and added in the form of a plug-in. The externally generated image analysis model or image recognition algorithm is generated by a deep learning server outside system 100, and may be generated by using image data from image providing device 112 and/or by learning separate image data irrelevant thereto. Since the externally generated image analysis model or image recognition algorithm is added, the image analysis and recognition rate of each image analyzing server of image analyzing server cluster 130 can be further improved.

Meanwhile, the image analysis model generated by deep learning server 190 may be provided to other systems outside system 100. This image analysis model or image recognition algorithm is generated by deep learning server 190 analyzing a large amount of image data from image providing device 112, and may be used usefully in the other systems and applied as an independent application with economic value.

Parameter database 194 may be a database that stores setting values that can be changed by the user in generating a deep learning model for image identification. Parameter database 194 may store different setting values for each user. The setting values stored in parameter database 194 may include a target object, an object identification method, a type of a target image, a type of an output image, a training model and method, and a format of a display. System 100 according to an embodiment may determine setting values necessary for generating a deep learning model as default values, or may adaptively change setting values such as a target image type and an output image type.

In an embodiment, system 100 may provide a deep learning user interface of a user-customized image identification deep learning platform, and the user may use user device 110 to access the deep learning user interface. System 100 may further include a server for providing the deep learning user interface. The server for providing the deep learning user interface may be configured in the form of a web server, but may not be limited thereto.

Even the user who does not have expertise in deep learning can easily perform uploading of an image including a desired object to be identified, labelling of the desired object to be identified from the image, and generation and updating of a deep learning model through the deep learning user interface provided by system 100. Also, the deep learning user interface provided by system 100 may provide various options for selecting, for example, the type of a deep learning result image, and the user may set a deep learning result to be provided by simply selecting a desired option. Option values provided by the deep learning user interface may be stored in parameter database 194 as described above. For example, the option values may include selection of a target object, selection of an image type, selection of a deep learning model, selection of a learning dataset, and the like, but may not be limited thereto.

Although deep learning server 190 shown in FIG. 1 is configured as a single server, it may be configured as a deep learning server cluster, such as image analyzing server cluster 130, composed of a plurality of deep learning servers in some embodiments. In this case, a routing device may be needed to allocate original image data from image database 140 to a suitable deep learning server according to characteristics of the corresponding channel, and this process may be performed by routing server 120 or a non-illustrated separate routing device. Further, a routing device may be needed to allocate operations of a plurality of users who accesses through a deep learning platform web interface to a specific deep learning server among a plurality of deep learning servers in the deep learning server cluster, and this process may be performed by I/O server 170 or a non-illustrated separate routing device.

System 100 may be further equipped with deep learning server 190 and learning data result database 192 that stores an image analysis model or image recognition algorithm as a result of learning, and, thus, the efficiency and performance in image analysis and object recognition of each image analyzing server of image analyzing server cluster 130 can be improved and a plurality of external users can participate in a learning task of deep learning server 190 or perform object labelling through the deep learning platform web interface for the plurality of external users. Meanwhile, deep learning server 190 is configured as a plurality of deep learning server clusters, and, thus, a more accurate image analysis model or image recognition algorithm can be generated by a deep learning server suitable for each channel and/or image through parallel analysis and learning. As a result, the accuracy and efficiency in image analysis and object recognition of the image analyzing servers included in image analyzing server cluster 130 can be further improved.

In some embodiments, deep learning server 190 may allow a labelling operator to perform labelling through the deep learning user interface. In other words, the labelling operator may access system 100 through the deep learning user interface and perform labelling of an object in image data being analyzed and learned, and labelling of objects may be reflected in the image analysis model or image recognition algorithm and stored together in learning data result database 192. Also, deep learning server 190 may provide an environment in which a plurality of labelling operators can access simultaneously and perform operations simultaneously.

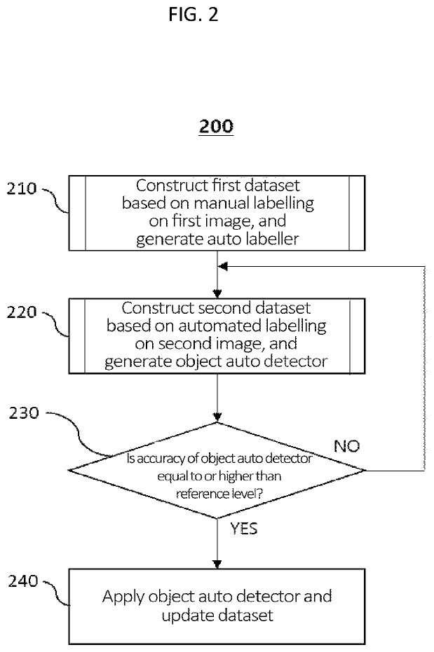

FIG. 2 shows an example of a process 200 for generating a user-customized image identification deep learning model according to at least some embodiments of the present disclosure. User-customized image identification deep learning system 100 of the present disclosure can provide a deep learning user interface that enables even a user without expertise in deep learning to generate a user-customized image identification deep learning model capable of identifying a desired object. A process 200 shown in FIG. 2 may include one or more operations, functions or actions as illustrated by blocks 210, 220, 230 and 240. The operations schematically illustrated in FIG. 2 are provided by way of example only, and some of the operations may be optional, may be combined into fewer operations, or may be expanded to additional operations without departing from the spirit of the disclosed embodiment. The blocks of process 200 shown in FIG. 2 may have identical or similar functions or actions to those described above with reference to FIG. 1, but are not limited thereto. Further, in a non-limiting embodiment, process 200 shown in FIG. 2 may be performed by system 100 shown in FIG. 1. Therefore, the blocks of process 200 shown in FIG. 2 will be described below in association with the components used in system 100 shown in FIG. 1.

Referring to FIG. 2, process 200 may begin in block 210 where a first dataset is constructed based on a manual labelling operation on a first image and generate an auto labeler.

In block 210, system 100 may perform manual labelling on at least one first image including an object to be identified by the user and generate a deep learning model based on a first dataset composed of manually labelled first images. In other words, system 100 may generate the deep learning model based on the manually labelled first dataset. The first image may be input by the user.

Manual labelling refers to an operation in which a user directly selects a desired object to be identified in an image and sets annotation information. System 100 may provide a deep learning user interface for manual labelling, which will be described later in detail. Process 200 may continue to block 220 where the deep learning model is updated using the deep learning model generated in block 210.

In block 220, system 100 may perform automated labelling on at least one second image by using the deep learning model generated based on the first dataset and update the deep learning model based on feedback on the automatically labelled second image. In other words, system 100 may generate a second dataset based on a result of feedback on the automatically labelled second image and generate a new deep learning model based on the first dataset and/or the second dataset. The second image may be input by the user.

Automated labelling refers to an operation in which a system automatically performs labelling without the intervention of a user by using a deep learning model based on a first dataset constructed by manual labelling. The user may perform feedback on a result of automated labelling on the second image, and system 100 may update the deep learning model based on the user's feedback. System 100 may provide a deep learning user interface for user feedback on automated labelling, which will be described later in detail. Process 200 may continue to block 230 where the validity of the deep learning model is checked by using the deep learning model updated in block 220.

In block 230, system 100 may check whether the accuracy of the deep learning model updated in block 220 is equal to or higher than a reference level. The accuracy of the deep learning model can be determined by the type of an identified object and whether the area of the object is accurately predicted. In an embodiment, the accuracy of the deep learning model may be measured by the indicators of Intersection Over Union (IoU), Precision, Recal, Average Precision (AP), Mean Average Precision (mAP), and Frame Per Second (PFS).

When the accuracy checked in block 230 is equal to or higher than the reference level, process 200 may continue to block 240 where the deep learning model is provided to the user, and when it is equal to or lower than the first reference level, process 200 may return back to block 220 where a dataset for updating the deep learning model is added. In some embodiments, when the accuracy checked in block 230 is equal to or lower than the second reference level lower than the first reference level, process 200 may return back to block 210 where a manual labelling operation is added. In block 240, system 100 may provide a deep learning user interface capable of providing the user with a deep learning model having an accuracy equal to or higher than the reference level.

In the present disclosure, the deep learning model generated in block 220 performs automated labelling on other images in block 230. Thus, it may be referred to as an auto labeler. Since the deep learning model updated in block 230 can be provided to a user, it may also be referred to as a customizing deep-learning model or an object auto detector. Also, the deep learning models generated in blocks 210 and 220 may be referred to as a first deep learning model and a second deep learning model, respectively, in the order of time series.

In some embodiments, system 100 may additionally perform the manual labelling operation of block 210 or the automated labeling and feedback operation of block 220 in order to further increase the accuracy of the deep learning model whose accuracy is equal to or higher than the reference level.

In this embodiment, for example the first image for the manual labeling is further included to update the first dataset, and the automatic labeler may also be updated based on the updated first dataset. In addition, the second images may be further added, and automatic labeling and feedback on the additional second images may also be performed by the updated automatic labeler. When additional labelling operations are performed, datasets are added, which can lead to improved accuracy of the deep learning model.

As such, the present disclosure establishes a process of generating a deep learning model so that even a user without expertise in deep learning can directly generate a user-customized image identification deep learning model for identifying a desired object to be identified. For example, in user-customized image identification deep learning system 100, the process of generating a user-customized deep learning model may be established by (1) the manual labelling operation (block 210), (2) the automated labelling and feedback operation (block 220), (3) the verification of the accuracy of the deep learning model (block 230). In some embodiments, system 100 of the present disclosure may further include (4) the addition of a labelling operation to improve accuracy. Hereinafter, details of the process and deep learning user interface provided by system 100 will be described.

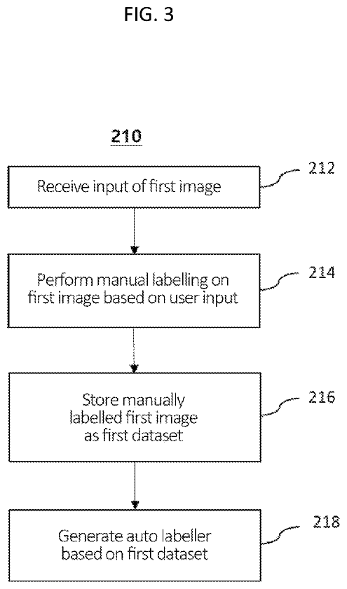

FIG. 3 shows a specific process of a manual labelling operation that is performed in the user-customized image identification deep learning system according to at least some embodiments of the present disclosure. The process shown in FIG. 3 may be a specific process of block 210 included in process 200. FIG. 4 and FIG. 5 show examples of a user interface to which the user-customized image identification deep learning system provides a manual labelling operation according to at least some embodiments of the present disclosure. The user interface provided by system 100 to user device 110 to receive a user input in performing a process 210 shown in FIG. 3 will be described with reference to FIG. 4 and FIG. 5.

Process 210 shown in FIG. 3 may include one or more operations, functions or actions as illustrated by blocks 212, 214, 216 and 218. The operations schematically illustrated in FIG. 3 are provided by way of example only, and some of the operations may be optional, may be combined into fewer operations, or may be expanded to additional operations without departing from the spirit of the disclosed embodiment.

Referring to FIG. 3, process 210 may begin in block 212 where system 100 receives at least one first image including an object that the user wants to identify. System 100 may provide a user interface to allow the user to perform process 210 on user device 110. At least a part of the user interface may be implemented through an application program installed on user device 110. That is, the user can develop and use a user-customized image identification deep learning model by using various convenient graphic user interfaces (GUIs) executed through the application program installed on user device 110.

In some embodiments, the user interface provided by system 100 of the present disclosure may be implemented through a web browser application executed on user device 110. Here, the web browser is a program that enables the user to use the World Wide Web (WWW) service and refers to a program that receives and displays hypertext written in hypertext mark-up language (HTML), and may include, for example, Netscape, Explorer, Chrome, etc.

Referring to FIG. 4, a user interface 400 may include an image upload button 410 that uploads an image, an image display region 420 that displays the uploaded image, labelling tools 430 that displays tools configured to enable the user to label the image, and an auto labeler generation request button 440 that generates an auto labeler based on the manually labelled image.

The user may upload a first image to system 100 through image upload button 410 displayed on user interface 400. For example, user device 110 may connect a channel as a path for providing an input image to system 100 (or routing server 120) based on a user input to image upload button 410. For another example, an image file stored in user device 110 may be directly transmitted to system 100. The first image provided to system 100 may be provided to deep learning server 190 through image database 140.

Process 210 may continue to block 212 where, upon receipt of the first image, a manual labelling operation is performed based on the user input on the first image. In block 212, system 100 may provide a deep learning user interface that enables the user to directly perform a manual labelling operation via user device 110. Referring to FIG. 4, the user may perform labelling on the uploaded image displayed on display region 420 included in user interface 400. The user may perform labelling by using labelling tools 430 included in user interface 400. Labelling tools 430 may include an object setting button 431 that sets an object that the user wants to identify, an annotation setting button 432 that sets an annotation on an object whose area is set, a deletion button 433 that deletes an image, and a storage button 434 that stores a labelled image as a dataset.

Although an object area 421 is shown as a circle in FIG. 4, areas of various shapes, such as rectangle and free polygon, and various sizes may be used. The annotation refers to, for example, giving a concept, such as type or name, for distinguishing an object to be identified. For example, an annotation 422 such as “vascular disorder” may be set as a disease name for object area 421 set in a chest X-ray image displayed on display region 420 of FIG. 4. The user may select and store an image to be used as a dataset for deep learning by using deletion button 433 or storage button 434.

In some embodiments, labelling may be performed to a plurality of objects in each first image. For example, an annotation such as “tuberculosis” may be set for another object area. In such an embodiment, system 100 may optionally further perform an operation to remove duplicate labelling through comparison between the annotated objects.

In an additional embodiment, user interface 400 may provide at least one of shortcut keys for tools displayed in labelling tools 430 and shortcut keys for commands such as drag and drop, zoom in and zoom out, and copy and paste.

When manual labelling on the first image is performed by the user, process 210 sequentially continues block 216 where a result of manual labelling is stored as a first dataset and block 218 where an auto labeler is generated based on the first dataset. The auto labeler (or first deep learning model) may include a task capable of performing deep learning on other data based on the first dataset.

Referring to FIG. 4, in response to an input to storage button 434 included in user interface 400, system 100 may store the image manually labelled by the user as a dataset. In response to an input to auto labeler generation request button 440 (or auto labeler generation button) included in user interface 400 to generate an auto labeler based on a manually labelled image, a deep learning model can be generated based on labelled images stored as a dataset. In other words, when system 100 receives a user input to storage button 434 of user interface 400, it may store a manually labelled image as a first dataset, and when system 100 receives a user input to auto labeler generation request button 440, it may generate a first deep learning model based on the manually labelled first dataset.

In an additional embodiment, system 100 may provide a user interface 500 capable of adding a first dataset separately from user interface 400. User interface 500 may be a user interface that can be seen when the user accesses again the user-customized image identification deep learning interface provided by system 100 after the user stores the first dataset through user interface 400 included in the user-customized image identification deep learning interface. In other words, it may be a user interface when the user stores additional data in the stored first dataset.

Referring to FIG. 5, user interface 500 may include a first dataset display region 510, an image display region 520, and an image addition button 530. First dataset display region 510 may display a list of a stored first dataset. Image display region 520 may display a labelled image of data selected from the list displayed on first dataset display region 510.

When the user makes a user input to image addition button 530 to add data, system 100 may provide user device 110 with user interface 400 of FIG. 4 for constructing a first dataset. That is, the user can optionally add the first dataset.

User interface 500 may include an auto labeler generation request button 540, and when a user input to auto labeler generation request button 540 is detected, system 100 may generate an auto labeler based on the currently constructed first dataset. That is, system 100 according to an embodiment of the present disclosure may further have an opportunity to improve the accuracy of the auto labeler by adding the first dataset.

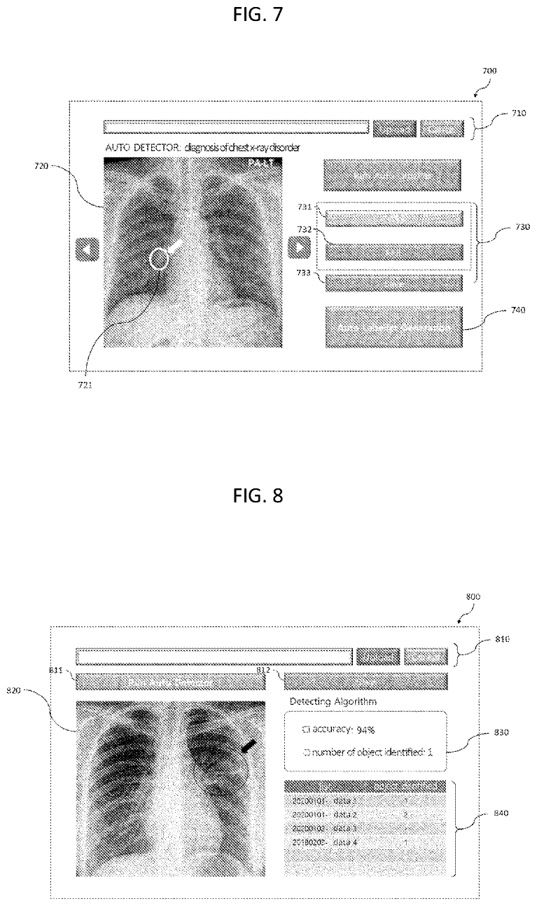

FIG. 6 shows a process of an automated labelling operation that is performed in the user-customized image identification deep learning system according to at least some embodiments of the present disclosure. The process shown in FIG. 6 may be a specific process of block 230 shown in FIG. 2. FIG. 7 shows an example of a user interface to which the user-customized image identification deep learning system provides an automated labelling operation according to at least some embodiments of the present disclosure. The user interface provided by system 100 to user device 110 to receive a user input in performing a process 220 shown in FIG. 6 will be described with reference to FIG. 7. Process 220 shown in FIG. 6 may include one or more operations, functions or actions as illustrated by blocks 222, 224, 226 and 228. The operations schematically illustrated in FIG. 5 are provided by way of example only, and some of the operations may be optional, may be combined into fewer operations, or may be expanded to additional operations without departing from the spirit of the disclosed embodiment.

Referring to FIG. 6, process 220 may begin in block 222 where system 100 receives at least one second image including an object that the user wants to identify. Referring to FIG. 7, a user interface 700 may include an image upload button 710, an image display region 720 that displays a result of automated labelling on an uploaded image with an object area 721, and a feedback input buttons 730 that inputs feedback on the result of automated labelling.

Process 220 may continue to block 224 where, upon receipt of the second image, an automated labelling operation is performed to the second image. System 100 may provide a user interface to allow the user to perform process 220 on user device 110. At least a part of the user interface may be implemented through an application program installed on user device 110. That is, the user can develop and use a user-customized image identification deep learning model by using various convenient graphic user interfaces (GUIs) executed through the application program installed on user device 110.

The user may upload a second image to system 100 through image upload button 710 displayed on user interface 700. For example, user device 110 may connect a channel as a path for providing an input image to system 100 (or routing server 120) based on a user input to image upload button 710. For another example, an image stored in user device 110 may be directly transmitted to system 100. The second image provided to system 100 may be provided to deep learning server 190 through image database 140.

Process 220 may continue to block 224 where, upon input of the second image, an automated labelling operation is performed to the second image.

In block 224, whenever the user inputs each second image, system 100 according to an embodiment may perform automated labelling on each second image by using the auto labeler generated based on the first dataset. In another embodiment, after the user inputs a second image and then makes an input to perform automated labelling, system 100 may perform a labelling operation on the second image by using the auto labeler based on the first dataset.

System 100 may provide a result of automated labelling on the second image to the user through user device 110. Referring to FIG. 7, the result of automated labelling may be displayed on image display region 720 of user interface 700.

Then, process 220 may continue to block 226 where the second image automatically labelled based on the user input is optionally stored as a second dataset.

In block 226, referring to FIG. 7, the user may input feedback on the automatically labelled image displayed on image display region 720 through feedback input buttons 730 included in user interface 700. Feedback input buttons 730 may include a pass button 732 for when the result of automated labelling on the second image is correct, i.e., when object identification is performed correctly, and a fail button 732 for when the result of automated labelling on the second image is not correct. An image in which object identification is successful and an image in which object identification has failed may be classified according to a user input to buttons 731 and 732.

In an embodiment, the second image to which it is determined that object identification has been correctly performed may be stored as a second dataset by pressing pass button 732. In an additional embodiment, the second image to which it is determined that object identification has not been performed correctly may be used as a dataset for generating a deep learning model through a manual labelling operation.

In an additional embodiment, user interface 700 may further include a storage button 733 that requests storage of the automatically labelled second image as a second dataset.

In an additional embodiment, as for an image on which a result of automated labelling is not accurate, a user interface that provides manual labelling on the image may be provided. The user may set an object area in the image or modify annotation information. In other words, user device 110 may be provided with a manual labelling tool for manual labelling on a second image to which it is determined that object identification has not been performed correctly. The manually labelled second image may be included in the first dataset, and the first dataset may be updated, including the manually labelled second image.

In block 228, an object auto detector may be generated based on the first dataset and the second dataset. That is, system 100 may update the deep learning model (auto labeler) by using the manually labelled first dataset and the automatically labelled second dataset.

Referring to FIG. 7, a deep learning model may be generated based on the labelled image stored as a dataset in response to an object auto detector generator 740 included in user interface 700 and configured to generates a second deep learning model based on a result of user feedback. In other words, when system 100 receives a user input to storage button 733 of user interface 700, it may store an automatically labelled image with secured accuracy according to a result of feedback as a second dataset, and when system 100 receives a user input to a second deep learning model generator 740, it may generate a second deep learning model based on the first dataset and the second dataset.

User-customized image identification deep learning system 100 of the present disclosure may provide a user interface that enables the user to use the object auto detector (or second deep learning model) generated based on the first dataset and the second dataset. However, when the accuracy of the second deep learning model is equal to or lower than a reference level, system 100 may request the user to add a manually or automatically labelled dataset. FIG. 8 shows a specific example of a deep learning user interface provided by user-customized image identification deep learning system 100 and displayed on a user device according to at least some embodiments of the present disclosure.

Referring to FIG. 8, a user interface 800 may display an applied image upload button 810, a result image display region 820, a result information display region 830, and a list 840.

The user may use the object auto detector generated by him or herself through user interface 800 provided to system 100. For example, the user may input an image to which the user wants to perform object recognition through upload button 810 included in user interface 800. In an embodiment, when the image is input, a result of object recognition performed on the image input by using the object auto detector may be simultaneously displayed on result image display region 820. However, in another embodiment, when the user makes an input to a detector execution button 811 included in user interface 800, a result of object recognition performed on the image input by using the object auto detector may be displayed on result image display region 820.

In an embodiment, the user may store an image in which an object is identified (or a labelled image) as a dataset for improving the performance of the object auto detector (or deep learning model) by making an input to storage button 812. The image stored in user interface 800 may be stored as a second dataset or a separate dataset.

Result information display region 830 may display the accuracy of the object auto detector and the number of cases where an object in a current image is identified. List 840 may show a list of images identified through user interface 800 and the number of objects identified for each image.

User-customized image identification deep learning system 100 according to various embodiments as described above establishes a simple process of generating a deep learning model, and provides an intuitive, natural and easy interaction in performing feedback on image input, manual labelling and automated labelling required for the above-described operations. Therefore, even a user without expertise in deep learning can have an opportunity to directly generate and use a user-customized image identification deep learning model for identifying a desired object to be identified.

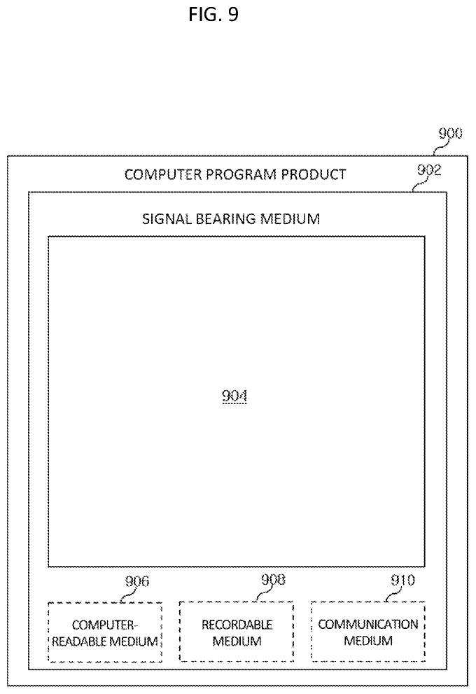

FIG. 9 shows an example of a computer program product 900 configured to operate a system for generating a user-customized image identification deep learning model according to at least some embodiments of the present disclosure. The computer program product is provided by using a signal bearing medium 902. In some embodiments, one or more signal bearing mediums 902 of computer program product 900 may include at least one instruction 904, a computer-readable medium 906, a recordable medium 908 and/or a communication medium 910.

Instructions 904 included in signal bearing medium 902 may be executed by, for example, one or more computing devices included in user-customized image identification deep learning system 100 or user device 110 shown in FIG. 1. Instructions 904 may include at least one of an instruction to receive at least one first image in response to a request from the user device by using the one or more computing devices, an instruction to perform manual labelling based on a user input on the at least one first image from the user device and store the manually labelled at least one first image as a first dataset, an instruction to generate a first deep learning model based on the first dataset, an instruction to receive at least one second image in response to a request from the user device, an instruction to perform automated labeling on the at least one second image by using the first deep learning model, an instruction to store at least one of the automatically labelled at least one second image as a second dataset based on a result of feedback on the automatically labelled at least one second image from the user device, and an instruction to generate a second deep learning model based on the first dataset and the second dataset.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The claimed subject matter is not limited in scope to the particular implementations described herein. For example, some implementations may be in hardware, such as employed to operate on a device or combination of devices, for example, whereas other implementations may be in software and/or firmware. Likewise, although claimed subject matter is not limited in scope in this respect, some implementations may include one or more articles, such as a signal bearing medium, a storage medium and/or storage media. This storage media, such as CD-ROMs, computer disks, flash memory, or the like, for example, may have instructions stored thereon, that, when executed by a computing device, such as a computing system, computing platform, or other system, for example, may result in execution of a processor in accordance with the claimed subject matter, such as one of the implementations previously described, for example. As one possibility, a computing device may include one or more processing units or processors, one or more input/output devices, such as a display, a keyboard and/or a mouse, and one or more memories, such as static random access memory, dynamic random access memory, flash memory, and/or a hard drive.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative example of the subject matter described herein applies regardless of the particular type of signal bearing medium used to actually carry out the distribution.

While certain example techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

Throughout this document, the term “connected to” may be used to designate a connection or coupling of one element to another element and includes both an element being “directly connected to” another element and an element being “electronically connected to” another element via another element. Through the whole document, the term “on” that is used to designate a position of one element with respect to another element includes both a case that the one element is adjacent to the other element and a case that any other element exists between these two elements. Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. An operation method of a user-customized image identification deep learning system, comprising:
receiving at least one first image in response to a request from a user device;
performing manual labelling based on a user input on the at least one first image from the user device and storing the manually labelled at least one first image as a first dataset;
generating a first deep learning model based on the first dataset;
receiving at least one second image in response to a request from the user device;
performing automated labelling on the at least one second image by using the first deep learning model;
storing at least one of the automatically labelled at least one second image as a second dataset based on a result of feedback on the automatically labelled at least one second image from the user device; and
generating a second deep learning model based on the first dataset and the second dataset.

2. The operation method of a user-customized image identification deep learning system of claim 1, further comprising:
measuring an accuracy of the second deep learning model.

3. The operation method of a user-customized image identification deep learning system of claim 2, further comprising:
updating the first dataset or the second dataset when the accuracy of the second deep learning model is measured to be equal to or lower than a reference level.

4. The operation method of a user-customized image identification deep learning system of claim 1, further comprising:
providing the user device with a first user interface including an image input button configured to receive the at least one first image, an image display region configured to display the at least one first image, a labelling tool configured to provide manual labelling to each of the at least one first image, and a storage button configured to request storage of the manually labelled at least one first image as a first dataset.

5. The operation method of a user-customized image identification deep learning system of claim 4,
wherein receiving at least one first image includes receiving information about an access route to an image providing device.

6. The operation method of a user-customized image identification deep learning system of claim 4,
wherein receiving at least one first image includes receiving an image stored in the user device.

7. The operation method of a user-customized image identification deep learning system of claim 1, further comprising:
providing the user device with a first user interface including an image input button configured to receive the at least one second image, an image display region configured to display the automatically labelled at least one second image, and a feedback input button configured to receive feedback on the automatically labelled at least one second image.

8. The operation method of a user-customized image identification deep learning system of claim 2, further comprising:
providing a third user interface including an image input button configured to receive a third image, and an image identification request button configured to request application of the second deep learning model to the third image.

9. The operation method of a user-customized image identification deep learning system of claim 8,
wherein the third user interface further includes an accuracy display region configured to display the accuracy of the second deep learning model.

10. A computer-readable storage medium that stores a computer program for developing a user-customized image identification deep learning model,
wherein the computer program includes one or more instructions to be executed by one or more computing devices in a user-customized image identification deep learning system, and the one or more instructions include:
an instruction to receive at least one first image in response to a request from a user device;
an instruction to perform manual labelling based on a user input on the at least one first image from the user device and store the manually labelled at least one first image as a first dataset;
an instruction to generate a first deep learning model based on the first dataset;
an instruction to receive at least one second image in response to a request from the user device;
an instruction to perform automated labelling on the at least one second image by using the first deep learning model;
an instruction to store at least one of the automatically labelled at least one second image as a second dataset based on a result of feedback on the automatically labelled at least one second image from the user device; and
an instruction to generate a second deep learning model based on the first dataset and the second dataset.

11. The computer-readable storage medium that stores a computer program for developing a user-customized image identification deep learning model of claim 10,
wherein the one or more instructions further include:
an instruction to measure an accuracy of the second deep learning model.

12. The computer-readable storage medium that stores a computer program for developing a user-customized image identification deep learning model of claim 11,
wherein the one or more instructions further include:
an instruction to update the first dataset or the second dataset when the accuracy of the second deep learning model is measured to be equal to or lower than a reference level.

13. The computer-readable storage medium that stores a computer program for developing a user-customized image identification deep learning model of claim 10,
wherein the one or more instructions further include:
an instruction to provide the user device with a first user interface including an image input button configured to receive the at least one first image, an image display region configured to display the at least one first image, a labelling tool configured to provide manual labelling to each of the at least one first image, and a storage button configured to request storage of the manually labelled at least one first image as a first dataset.

14. The computer-readable storage medium that stores a computer program for developing a user-customized image identification deep learning model of claim 13,
wherein the instruction to receive at least one first image includes an instruction to receive information about an access route to an image providing device.

15. The computer-readable storage medium that stores a computer program for developing a user-customized image identification deep learning model of claim 13,
wherein the instruction to receive at least one first image includes an instruction to receive an image stored in the user device.

16. The computer-readable storage medium that stores a computer program for developing a user-customized image identification deep learning model of claim 10,
wherein the one or more instructions further include:
an instruction to provide the user device with a first user interface including an image input button configured to receive the at least one second image, an image display region configured to display the automatically labelled at least one second image, and a feedback input button configured to receive feedback on the automatically labelled at least one second image.

17. The computer-readable storage medium that stores a computer program for developing a user-customized image identification deep learning model of claim 11,
wherein the one or more instructions further include:
an instruction to provide a third user interface including an image input button configured to receive a third image, and an image identification request button configured to request application of the second deep learning model to the third image.

18. The computer-readable storage medium that stores a computer program for developing a user-customized image identification deep learning model of claim 17,
wherein the third user interface further includes an accuracy display region configured to display the accuracy of the second deep learning model.
